(19) United States
(12) Patent Application Publication
(10) Pub. No.: US 2026/0028982 A1
(43) Pub. Date: Jan. 29, 2026

(54) A ONE-WAY PUMPING DEVICE AND ITS APPLICATION DEVICE

(71) Applicant: Jianjun MI, Laixi City (CN)

(72) Inventor: Jianjun MI, Laixi City (CN)

(21) Appl. No.: 19/350,441

(22) Filed: Oct. 6, 2025

Related U.S. Application Data
(63) Continuation of application No. PCT/CN2024/096919, filed on Jun. 2, 2024.

(30) Foreign Application Priority Data
Apr. 8, 2023 (CN) 202310368191.6

(51) Int. Cl. F04B 53/10; A43B 13/20; A61M 60/117; A61M 60/268; A61M 60/416; A61M 60/894; F02B 33/32

(52) U.S. Cl. CPC F04B 53/1077; A61M 60/117; A61M 60/268; A61M 60/416; A61M 60/894; F04B 53/1072; A43B 13/203; F02B 33/32

(57) ABSTRACT

A one-way pumping device and its application devices, so as to enhance the resistance difference effect between the forward direction and the reverse direction of the one-way pumping device and realize a structure applicable to more application devices. To achieve the above purpose, this application provides the following technical solutions: a one-way pumping device, an artificial heart, and an internal combustion engine exhaust gas supercharging device.

### CROSS-REFERENCE TO THE RELATED APPLICATIONS

This application is a Continuation of International Application No. PCT/CN2024/096919, filed on June. 2, 2024, which is based upon and claims priority to Chinese Patent Application No. 202310368191.6, filed on April. 8, 2023, the entire contents of which are incorporated herein by reference.

### TECHNICAL FIELD

This application relates to the technical fields of one-way pumping technology, artificial heart technology, and internal combustion engine technology, and specifically to a one-way pumping device and its application device.

### BACKGROUND TECHNOLOGY

In human production and daily life, various types of pumping devices are needed to transport fluids to required locations. One-way pumping devices in the existing technology require the assistance of one-way valves or one-way flaps. One-way valves can only allow intermittent fluid flow, and one-way flaps must have continuously bending structures to form intermittent fluid flow, which are prone to malfunctions and thus affect production and daily life. For example, if one-way flaps are used in an artificial heart, long-term use may lead to reduced elasticity or breakage of the one-way flaps. Additionally, the existing turbocharging systems for internal combustion engines have problems such as complex structures and high process requirements, which call for improvements.

### SUMMARY OF THE INVENTION

The purpose of this application is to provide a one-way pumping device and its application device, so as to enhance the resistance difference between the forward and reverse directions of the one-way pumping device and enable the structure to be applicable to more application scenarios.

To achieve the above purpose, this application adopts the following technical solutions: A one-way pumping device, which includes two one-way pipes 5 and a deformable pipe 1 connected between the two one-way pipes 5. The two one-way pipes 5 are arranged in the same direction. The deformable pipe 1 has radial and/or axial elasticity, and can change its shape under the action of an external force to alter its internal volume. Cooperating with the two one-way pipes 5, it enables the fluid passing through the interior of the one-way pumping device to flow in a single direction. The one-way pipe 5 includes at least one one-way pipe unit, and each one-way pipe unit consists of a main pipe wall 51, a shunt pipe 54, and a bracket 55. The main pipe wall 51 is in the shape of a hollow cone with a central through-hole, having a thin end and a thick end. The shunt pipe 54 is in the shape of a horn with a central through-hole, also having a thin end and a thick end. The shunt pipe 54 is suspended and fixed inside the main pipe wall 51 via the bracket 55. The shunt pipe 54 and the main pipe wall 51 are arranged in the same corresponding direction and are coaxial. The central through-holes of the shunt pipe 54 and the main pipe wall 51 form a main channel 56, and a shunt channel 57 with a U-shaped cross-section is formed between the shunt pipe 54 and the main pipe wall 51.

The central through-holes of the one-way pipe units have different radial dimensions. The one-way pipe units are connected end-to-end in series according to the order of the radial dimensions of their central through-holes, so that a main channel 56 with gradually increasing or decreasing radial dimensions is formed inside the one-way pipe. A plurality of one-way pipe units are connected end-to-end in series in the same direction to form the main channel 56 of the one-way pipe 5. The one-way pipe 5 is provided with a forward inlet 53 and a reverse inlet 52 at its two ends, and the cross-sectional area of the forward inlet 53 is larger than that of the reverse inlet 52.

When an external force is applied, the pipe wall of the end of the deformable pipe 1 close to the outlet is more likely to deform than the pipe wall of the end close to the inlet. This can be achieved by changing the thickness of the pipe wall—for example, the thickness of the pipe wall at the end of the deformable pipe 1 close to the outlet is gradually increased compared to that at the end close to the inlet. It can also be realized through structural design, such as forming the pipe wall with corrugations of different sizes and/or shapes.

A housing 2 is further arranged on the periphery of the deformable pipe 1. A pressure chamber 4, which is a closed space, is formed between the housing 2 and the deformable pipe 1. At least one fluid pipe 3 is disposed on the pipe wall of the housing 2, and the fluid pipe 3 is in communication with the pressure chamber 4.

The housing 2 is either a rigid shell or has elasticity. When the housing 2 has elasticity, it is more difficult for the housing 2 to change its shape than the deformable pipe 1 under the action of the same external force; that is, the hardness of the housing 2 is greater than that of the deformable pipe 1, or the elastic force of the housing 2 is greater than that of the deformable pipe 1. When the pressure of the fluid in the pressure chamber 4 between the housing 2 and the deformable pipe 1 changes, the radial deformation amplitude of the deformable pipe 1 is larger than that of the housing 2.

The deformable pipe 1 is a corrugated pipe, and the cross-sectional area of the inlet of the deformable pipe 1 is smaller than that of the outlet.

The cross-section of the deformable pipe 1 is circular or elliptical; adopting an elliptical cross-section for the deformable pipe 1 is beneficial for increasing the contact area with the external environment, facilitating the side-by-side arrangement of multiple deformable pipes 1, and enabling shape changes of the deformable pipes 1.

An artificial heart includes an external part and an internal part. The internal part is used to partially or completely replace the function of the heart and serves as a blood pump. The external part is connected to the internal part via a pipeline to drive and control the internal part. It is characterized in that the blood pump adopts the one-way pumping device described in any one of the above, and the blood pump is in communication with the external part through a fluid pipe 3.

In the artificial heart, the external part is in communication with the fluid pipe 3. The external part generates pulsating pressure, which transmits power to the fluid in the pressure chamber 4 of the one-way pumping device through the fluid pipe 3, causing the fluid in the pressure chamber 4 to generate pulsating pressure. This pulsating pressure drives the deformable pipe 1 to change its shape, thereby altering its internal volume. The change in the internal volume of the deformable pipe 1, combined with the action of the one-way pipe 5, enables blood to flow unidirectionally inside the one-way pumping device.

The external part of the artificial heart is a hollow ball 6, and the hollow ball 6 is connected to the pressure chamber 4 between the inside of the housing 2 of the one-way pumping device and the deformable pipe 1 through the fluid pipe 3.

The external part includes a hollow ball 6, a driving pump 7, and a valve 8. The hollow ball 6 and the driving pump 7 are respectively connected in parallel to the fluid pipe 3 through the valve 8, and then connected to the pressure chamber 4 inside the deformable pipe 1 of the one-way pumping device.

The driving pump 7 includes a power supply 70 connected to a control board 71, the control board 71 connected to a motor 72, an output shaft of the motor 72 connected to a rotating wheel 73, the rotating wheel 73 movably connected to a connecting rod 74, the connecting rod 74 movably connected to a reciprocating rod 76, the reciprocating rod 76 connected to a diaphragm 77 through a sliding sleeve 79. The diaphragm 77 has elasticity, and the edge of the diaphragm 77 is fixedly arranged in a spherical pump chamber 78. The pump chamber 78 is connected to the fluid pipe 3. A closed cavity is formed between the diaphragm 77 and the side of the spherical pump chamber 78 facing the fluid pipe 3. An air hole 75 is arranged on the side of the spherical pump chamber 78 where the diaphragm 77 is located and which faces the reciprocating rod 76.

The driving pump 7 includes a power supply 70 connected to a control board 71, the control board 71 connected to an electromagnetic coil 10, one end of a reciprocating rod 76 extending into the electromagnetic coil 10, the reciprocating rod 76 connected to a diaphragm 77 through a sliding sleeve 79. The diaphragm 77 has elasticity, and the edge of the diaphragm 77 is fixedly arranged in a spherical pump chamber 78. The pump chamber 78 is connected to the fluid pipe 3. A closed cavity is formed between the diaphragm 77 and the side of the spherical pump chamber 78 facing the fluid pipe 3. An air hole 75 is arranged on the side of the spherical pump chamber 78 where the diaphragm 77 is located and which faces the reciprocating rod 76. At least the part of the reciprocating rod 76 inside the electromagnetic coil 10 has ferromagnetism.

The external part includes an airbag shoe 18. The airbag shoe 18 includes a shoe body 180 and an airbag 181 arranged under the shoe body 180. The airbag 181 is provided with an airbag port 182 at the heel position of the shoe body 180. The airbag 181 is squeezed and released to deform it, and the internal gas enters and exits through the airbag port 182. An airbag seal 183 is arranged on the shoe body 180 near the airbag port 182. The airbag 181 has elasticity. The fluid pipe 3 is inserted into the airbag port 182 to connect to the airbag 181, and the fluid pipe 3 is fixed with a pipe clip 184. The fluid pipe 3 extends upward along the human leg and is connected to the pressure chamber 4 of the one-way pumping device.

An exhaust gas supercharging device for an internal combustion engine. The internal combustion engine body is provided with an exhaust pipe 9. The one-way pumping device is arranged on the exhaust pipe 9. At least the deformable pipe 1 of the one-way pumping device is arranged inside the exhaust pipe 9. The one-way pipes 5 at both ends of the deformable pipe 1 extend out through the outer wall of the exhaust pipe 9. Both ends of the one-way pumping device are connected in series to the intake pipe of the internal combustion engine. The reverse inlets 52 at both ends of the one-way pumping device face the direction of the internal combustion engine body, and the forward inlets 53 face the direction of the intake port of the intake pipe of the internal combustion engine. At least one deformable pipe 1 is arranged.

The one-way pipes 5 at both ends of the one-way pumping device are arranged on the same side of the exhaust pipe 9 or arranged on the side of the exhaust pipe 9 at other angles.

In the intake manifold of the internal combustion engine, an intake filter, a vibration isolator, an intercooler, a throttle valve, and an intake manifold are sequentially arranged from the intake port, and the one-way pipe is arranged between the vibration isolator and the intercooler.

An exhaust gas supercharging device for an internal combustion engine. The internal combustion engine body is provided with an exhaust pipe 9. A plurality of the one-way pumping devices are arranged on the exhaust pipe 9. The deformable pipes 1 of the plurality of one-way pumping devices are arranged inside the exhaust pipe 9. The plurality of deformable pipes 1 are arranged in parallel along the axial direction (longitudinally) and/or radial direction of the exhaust pipe 9 inside the exhaust pipe 9. Both ends of the plurality of one-way pumping devices are respectively connected in series to different pipes of the internal combustion engine to supercharge the fluid inside them. It includes at least two of the following items: both ends of one of the one-way pumping devices are connected to the intake pipe of the internal combustion engine for supercharging the intake air; both ends of one of the one-way pumping devices are connected to the air pressure pipe of the vehicle for supercharging the air inside the air pressure pipe, which is used for braking, etc.; both ends of one of the one-way pumping devices are connected to the fuel pipe of the vehicle for driving the fuel flow to supply the internal combustion engine; both ends of one of the one-way pumping devices are connected to the oil pipe of the vehicle for driving the oil to the lubrication part. By arranging a plurality of one-way pumping devices and using the exhaust gas of the internal combustion engine, the kinetic energy of the exhaust gas is used to drive and supercharge different systems. Its working principle is the same as that described above in this application, and will not be repeated here. For the exhaust pipe 9 provided with a plurality of the one-way pumping devices, the order of the plurality of one-way pumping devices can be as follows: both ends of one of the one-way pumping devices are connected to the fuel pipe of the vehicle, and this one-way pumping device is arranged at the first position on the exhaust pipe 9 closest to the internal combustion engine body; both ends of one of the one-way pumping devices are connected to the intake pipe of the internal combustion engine, and this one-way pumping device is arranged at the second position on the exhaust pipe 9 away from the internal combustion engine body; both ends of one of the one-way pumping devices are connected to the air pressure pipe of the vehicle, and this one-way pumping device is arranged at the third position on the exhaust pipe 9 away from the internal combustion engine body; both ends of one of the one-way pumping devices are connected to the oil pipe of the vehicle, and this one-way pumping device is arranged at the fourth position on the exhaust pipe 9 away from the internal combustion engine body.

The beneficial effects of this application are as follows: By adopting the one-way pumping device described in this application, the one-way pumping function is realized through the combination of the one-way pipe structure and the deformable pipe. The artificial heart made by using the one-way pumping device has no rotating parts, simple structure, low noise, long service life, conforms to the original heartbeat attribute of the human body, and is beneficial to other organs. Compared with the existing turbocharging technology, the exhaust gas supercharging device for the internal combustion engine made by using the one-way pumping device has simple manufacturing process, low cost, and is applicable to fluid supercharging of other systems.

### BRIEF DESCRIPTION OF THE DRAWINGS

FIG. 1 is a schematic diagram of the external structure of a one-way pumping device according to Embodiment 1 of this application;

FIG. 2 is a cross-sectional schematic diagram of the one-way pumping device shown in FIG. 1;

FIG. 3 is a schematic diagram of the external structure of a one-way pumping device according to Embodiment 2 of this application;

FIG. 4 is a schematic diagram of the structure of the one-way pipe of the one-way pumping device of this application;

FIG. 5 is a cross-sectional schematic diagram of the one-way pipe unit of this application;

FIG. 6 is a schematic diagram of the reverse flow of fluid in the one-way pipe of this application;

FIG. 7 is a schematic diagram of the forward flow of fluid in the one-way pipe of this application;

FIG. 8 is a schematic diagram of the structure of a one-way pumping device according to Embodiment 3 of this application;

FIG. 9 is a cross-sectional schematic diagram of the one-way pumping device shown in FIG. 8;

FIG. 10 is a schematic diagram of the structure of an artificial heart according to Embodiment 4 of this application;

FIG. 11 is a schematic diagram of the structure of an artificial heart according to Embodiment 5 of this application;

FIG. 12 is a cross-sectional schematic diagram of the interior of one of the driving pumps of the artificial heart according to Embodiment 5 shown in FIG. 11;

FIG. 13 is a schematic diagram of the structure of a one-way pumping device according to Embodiment 6 of this application;

FIG. 14 is a cross-sectional schematic diagram of the interior of the second driving pump of the artificial heart according to Embodiment 5 shown in FIG. 11;

FIG. 15 is a schematic diagram of the structure of an exhaust gas supercharging device for an internal combustion engine of this application;

FIG. 16 is a partial cross-sectional schematic diagram of the exhaust pipe shown in FIG. 15;

FIG. 17 is a schematic diagram of the connection structure between one of the exhaust gas supercharging devices for the internal combustion engine and the internal combustion engine of this application;

FIG. 18 is a schematic diagram of the connection structure between the second exhaust gas supercharging device for the internal combustion engine and the internal combustion engine of this application;

FIG. 19 is a partial cross-sectional schematic diagram of the exhaust pipe according to Embodiment 6 of this application;

FIG. 20 is a partial cross-sectional schematic diagram of the exhaust pipe according to Embodiment 7 of this application;

FIG. 21 is a schematic diagram of the deformable pipes arranged in parallel inside the exhaust pipe according to Embodiment 8 of this application;

FIG. 22 is a cross-sectional schematic diagram of the airbag shoe according to Embodiment 9 of this application;

In the figures: 1—deformable pipe, 2—housing, 3—fluid pipe, 4—pressure chamber, 5—one-way pipe, 51—main pipe wall, 52—reverse inlet, 53—forward inlet, 54—shunt pipe, 55—bracket, 56—main channel, 57—shunt channel, 6—hollow ball, 7—driving pump, 71—control board, 72—motor, 73—rotating wheel, 74—connecting rod, 75—air hole, 76—reciprocating rod, 77—diaphragm, 78—pump chamber, 8—valve, 9—exhaust pipe, 10—electromagnetic coil, 11—bracket, 12—filter, 13—resonance pipe, 14—internal combustion engine body, 15—intercooler, 16—heat dissipation pipe, 17—throttle valve, 18—airbag shoe, 180—shoe body, 181—airbag, 182—airbag port, 183—airbag seal, 184—pipe clip.

### EMBODIMENTS

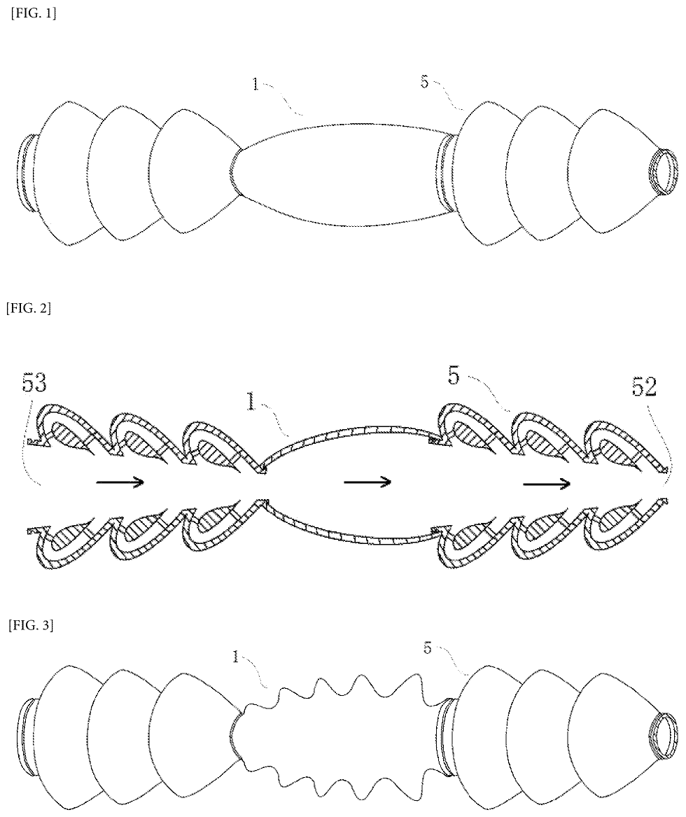

The application will be further elaborated below in conjunction with specific embodiments. FIG. 1 is a schematic diagram of the external structure of a one-way pumping device according to Embodiment 1 of this application; FIG. 2 is a cross-sectional schematic diagram of the one-way pumping device shown in FIG. 1; In FIG. 1, one-way pipes 5 are respectively arranged at both ends of the deformable pipe 1. The one-way pipe 5 is provided with a forward inlet 53 and a reverse inlet 52. Both ends of the deformable pipe 1 are respectively connected to the reverse inlet 52 of one one-way pipe 5 and the forward inlet 53 of another one-way pipe 5. The cross-sectional area of the forward inlet 53 of the one-way pipe 5 is larger than that of the reverse inlet 52. The deformable pipe 1 has elasticity and can change its internal volume under the action of an external force. When there is fluid such as gas or liquid inside the one-way pumping device, when the deformable pipe 1 is squeezed by an external force to reduce its internal volume, the internal fluid flows to the one-way pipes 5 at both ends. Since the resistance of the one-way pipe 5 to fluid flow from the forward inlet 53 to the reverse inlet 52 is smaller than that from the reverse inlet 52 to the forward inlet 53, the fluid inside the one-way pumping device flows in the direction from the forward inlet 53 to the reverse inlet 52. When the external force is removed and the deformable pipe 1 recovers by virtue of elasticity or the deformable pipe 1 is acted by an external force to increase its internal volume, similarly, due to the fact that the resistance of the one-way pipe 5 to fluid flow from the forward inlet 53 to the reverse inlet 52 is smaller than that from the reverse inlet 52 to the forward inlet 53, the fluid inside the one-way pumping device flows in the direction from the forward inlet 53 to the reverse inlet 52; It can be seen from FIG. 2 that the cross-sectional area of one end of the deformable pipe 1 is smaller than that of the other end. This arrangement is beneficial for the deformable pipe 1 to act on the internal fluid to make it flow from the end with the smaller cross-sectional area to the end with the larger cross-sectional area when the deformable pipe 1 works; The deformable pipe 1 is made of materials such as rubber or silica gel.

FIG. 3 is a schematic diagram of the external structure of a one-way pumping device according to Embodiment 2 of this application; This figure adopts a corrugated deformable pipe 1, which can make the deformable pipe 1 softer and more elastic, and can be made of harder materials, such as thin stainless steel.

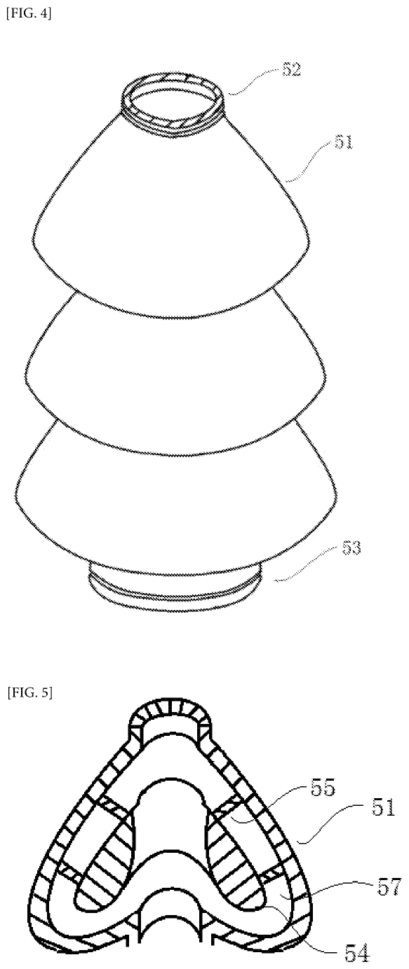

The structures of the one-way pipe unit and the one-way pipe 5 described in this application can be understood through FIG. 2, FIG. 3, FIG. 4, and FIG. 5. The difference from the structure of the prior art in FIG. 1 can be understood. The one-way pipe 5 of this application can be manufactured by processes such as casting, stamping and spinning, injection molding, and 3D printing. The shunt pipe wall of the shunt pipe 54 can be set as a hollow structure, for example, when the external dimension of the shunt pipe 54 is large and light weight is particularly required, refer to FIG. 4. FIG. 4 is a schematic diagram of the structure of the one-way pipe of the one-way pumping device of this application.

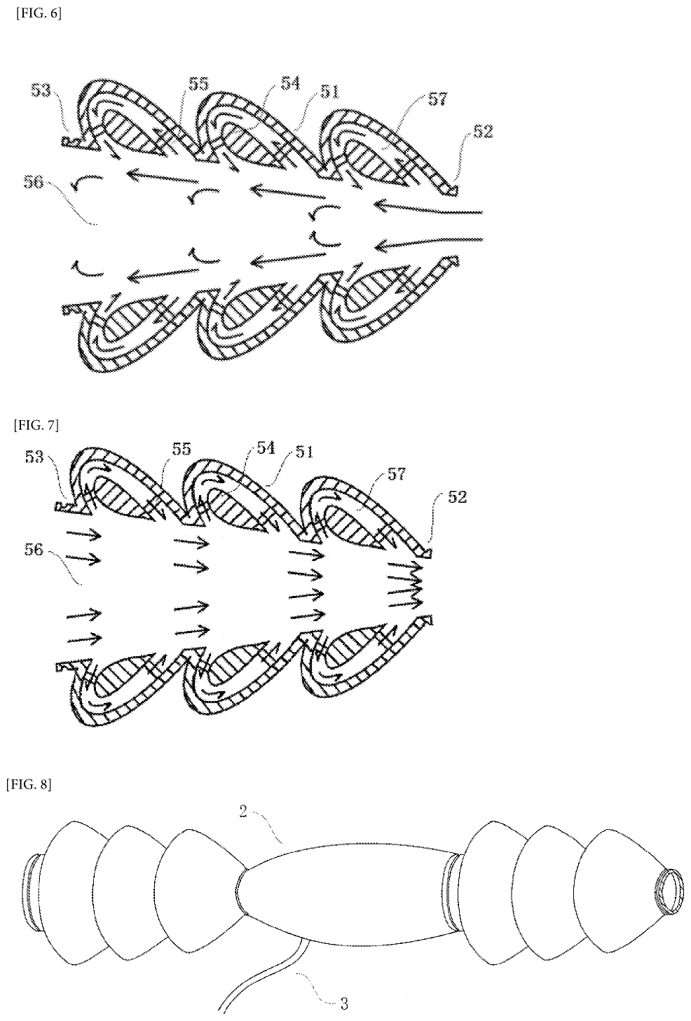

FIG. 6 is a schematic diagram of the reverse flow of fluid in the one-way pipe of this application; This figure shows that fluid enters the one-way pipe 5 from the reverse inlet 52, flows in the main channel 56 in the middle axis direction, and also flows from the shunt channel 57 between the shunt pipe 54 and the main pipe wall 51. When the two meet, they collide and rub against each other due to opposite directions, thereby offsetting the forward kinetic energy of the fluid and achieving the purpose of deceleration. Only the plane effect is shown in this figure, and the fluid in the main channel 56 and the shunt channels 57 on both sides meet. However, the one-way pipe 5 of this application is actually a three-dimensional structure, in which the fluid in the main channel 56 and the shunt channel 57 meet in the entire circumferential direction, resulting in a stronger interaction.

FIG. 7 is a schematic diagram of the forward flow of fluid in the one-way pipe of this application; This figure shows that fluid enters the one-way pipe 5 from the forward inlet 53, flows in the main channel 56 in the middle axis direction, and also flows from the shunt channel 57 between the shunt pipe 54 and the main pipe wall 51. When the two meet, they accelerate each other due to the same direction, achieving the purpose of acceleration. Only the plane effect is shown in this figure, and the fluid in the main channel 56 and the shunt channels 57 on both sides meet. However, the one-way pipe 5 of this application is a three-dimensional structure, in which the fluid in the main channel 56 and the shunt channel 57 meet in the entire circumferential direction, resulting in a stronger interaction.

This application adopts a variable-diameter one-way pipe 5 structure, that is, the cross-sectional area of the main channel 56 of the one-way pipe 5 gradually decreases along the direction from the forward inlet 53 to the reverse inlet 52. This arrangement is beneficial to further increase the resistance difference between the forward and reverse flows of the fluid inside the one-way pipe 5.

The deformable pipe 1 of this application adopts a variable-diameter structure, that is, the cross-sectional area of one end of the deformable pipe 1 is smaller than that of the other end. That is, the cross-sectional area of the left end of the deformable pipe 1 shown in FIG. 2 is smaller than that of the right end. This arrangement is adapted to the structure and arrangement direction of the variable-diameter one-way pipe 5 connected to both ends of the deformable pipe 1. That is, the left end of the deformable pipe 1 in FIG. 2 is connected to the reverse inlet 52 of the one-way pipe 5 at its left end, and the right end of the deformable pipe 1 is connected to the forward inlet 53 of the one-way pipe 5 at its right end. When the deformable pipe 1 is squeezed inward and expanded outward by force, the fluid inside the deformable pipe 1 flows from the left end with the smaller cross-sectional area to the right end with the larger cross-sectional area under the influence of the inclined surface of the deformable pipe 1.

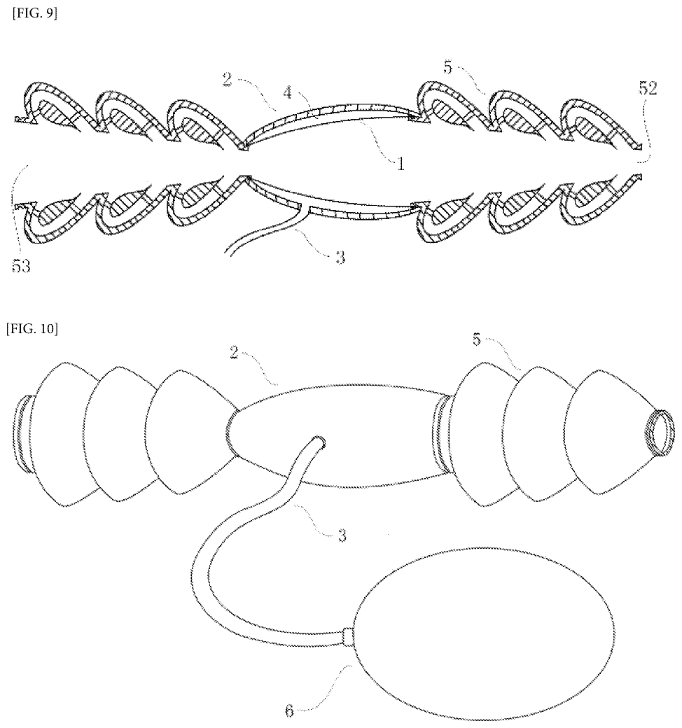

The pipe wall of the deformable pipe 1 can be set such that the thickness of the end close to the large cross-sectional area is smaller than that of the end close to the small cross-sectional area, that is, the thickness of the right end of the deformable pipe 1 shown in FIG. 9 is smaller than that of the left end; Therefore, when the deformable pipe 1 is subjected to the pressure and suction force of the fluid in the pressure chamber 4, the deformation amplitude of the right end of the deformable pipe 1 is larger than that of the left end, which is beneficial for the fluid inside the deformable pipe 1 to flow from the left end to the right end.

The deformable pipe 1 and the one-way pipe 5 shown in FIG. 1, FIG. 3, and FIG. 8 are connected in a straight line direction. The deformable pipe 1 and the one-way pipe 5 can also be connected in a bent direction, for example, the deformable pipe 1 and the one-way pipe 5 are connected at a right angle.

The deformable pipe 1 and the one-way pipe 5 shown in FIG. 1, FIG. 3, and FIG. 8 have a straight axis of the deformable pipe 1 itself. The axis of the deformable pipe 1 itself can be set to a curved shape. The axis of the one-way pipe 5 itself is straight, and the axis of the one-way pipe 5 itself can be set to a curved shape; For example, the axes of the deformable pipe 1 and the one-way pipe 5 are set to an arc shape, that is, the entire one-way pumping device is arc-shaped.

FIG. 8 is a schematic diagram of the structure of a one-way pumping device according to Embodiment 3 of this application; The one-way pumping device shown in this figure is provided with a housing 2 on the periphery of the deformable pipe 1. A pressure chamber 4 which is a closed space is formed between the housing 2 and the deformable pipe 1. At least one fluid pipe 3 is arranged on the pipe wall of the housing 2, and the fluid pipe 3 is connected to the pressure chamber 4; Its internal structure refers to FIG. 9. FIG. 9 is a cross-sectional schematic diagram of the one-way pumping device shown in FIG. 8; One-way pipes 5 are arranged at both ends of the deformable pipe 1 and the housing 2, and both ends of the deformable pipe 1 are respectively connected end-to-end with the two one-way pipes 5.

The housing 2 is a rigid shell or has elasticity; When the housing 2 has elasticity, under the action of the same external force, the housing 2 is more difficult to change its shape than the deformable pipe 1; When the housing 2 is a rigid shell, the outside conveys fluid into the pressure chamber 4 between the housing 2 and the deformable pipe 1 through the fluid pipe 3. The fluid is water, air, etc. The deformable pipe 1 is squeezed and deforms inward, reducing its internal volume and squeezing the fluid inside the deformable pipe 1 to flow. The outside draws the fluid out of the pressure chamber 4 between the housing 2 and the deformable pipe 1 through the fluid pipe 3. The deformable pipe 1 is attracted and deforms outward, increasing its internal volume and attracting the fluid inside the deformable pipe 1 to flow. The deformable pipe 1 and the one-way pipe 5 work together to make the fluid inside the entire one-way pumping device flow unidirectionally, that is, from the left end to the right end of the one-way pumping device shown in FIG. 8; When the housing 2 is an elastic housing 2, the outside conveys fluid into the pressure chamber 4 between the housing 2 and the deformable pipe 1 through the fluid pipe 3. The deformable pipe 1 and the housing 2 are squeezed, the housing 2 deforms outward, and the deformable pipe 1 deforms inward, reducing the internal volume of the deformable pipe 1 and squeezing the fluid inside the deformable pipe 1 to flow. The outside draws the fluid out of the pressure chamber 4 between the housing 2 and the deformable pipe 1 through the fluid pipe 3. The housing 2 deforms inward, and the deformable pipe 1 is attracted and deforms outward, increasing its internal volume and attracting the fluid inside the deformable pipe 1 to flow. The deformable pipe 1 and the one-way pipe 5 work together to make the fluid inside the entire one-way pumping device flow unidirectionally, that is, from the left end to the right end of the one-way pumping device shown in FIG. 8.

By setting the elastic deformation force of the housing 2 to be greater than that of the deformable pipe 1, when the fluid in the pressure chamber 4 increases or decreases, the deformation of the housing 2 is smaller than that of the deformable pipe 1.

Setting the housing 2 to have elasticity is beneficial to match the elastic deformation of the deformable pipe 1, that is, when the deformable pipe 1 deforms and causes axial and/or radial dimensional changes, the housing 2 also changes its axial and/or radial dimensions accordingly.

Setting the housing 2 to have elasticity, during the elastic deformation of the housing 2, the axial and radial dimensions of the housing 2 change, and the overall length of the one-way pumping device changes. During this change, the fluid inside the one-way pumping device moves pulsatingly along the axial direction of the one-way pumping device relative to the one-way pumping device, which is superimposed with the one-way pumping effect of the one-way pumping device itself, further enhancing the one-way pumping effect of the fluid inside the one-way pumping device.

FIG. 10 is a schematic diagram of the structure of an artificial heart according to Embodiment 4 of this application; The artificial heart includes an external part and an internal part. The internal part is used to partially or completely replace the function of the heart, and the internal part is a blood pump. The external part is connected to the internal part through a pipeline to drive and control the internal part. The blood pump adopts the one-way pumping device described above, and the blood pump is connected to the external part through a fluid pipe 3; The external part of the artificial heart is connected to the fluid pipe 3. The external part generates pulsating pressure, which transmits power to the fluid in the pressure chamber 4 of the one-way pumping device through the fluid pipe 3, causing the fluid in the pressure chamber 4 to generate pulsating pressure, driving the deformable pipe 1 to change its shape so as to change its internal volume. Under the joint action of the one-way pipe 5, the blood flows unidirectionally inside the one-way pumping device; The blood pump is connected in parallel or in series with blood vessels or the heart.

The external part of the artificial heart is a hollow ball 6. The internal space of the hollow ball 6 is connected to the pressure chamber 4 between the inside of the housing 2 of the one-way pumping device and the deformable pipe 1 through the fluid pipe 3. The fluid in the hollow ball 6, the fluid pipe 3, and the pressure chamber 4 is pure water.

The hollow ball 6 has elasticity, and its shape is changed by applying pressure through hand pinching, foot stepping, or other devices, and the hollow ball 6 recovers its shape by virtue of elasticity; The hollow ball 6 is made of rubber, silica gel, or a composite of rubber and elastic steel wire, for example, annular steel wire is cast into rubber.

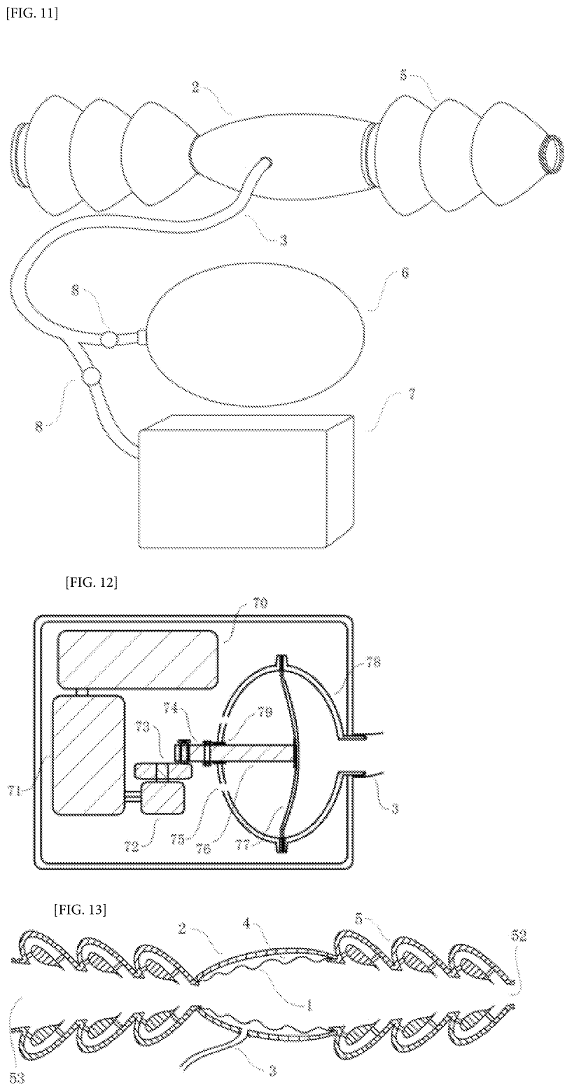

FIG. 11 is a schematic diagram of the structure of an artificial heart according to Embodiment 5 of this application; The artificial heart includes an external part and an internal part. The internal part is used to partially or completely replace the function of the heart, and the internal part is a blood pump. The external part is connected to the internal part through a pipeline to drive and control the internal part. The blood pump adopts the one-way pumping device described above, and the blood pump is connected to the external part through a fluid pipe 3. The external part includes a hollow ball 6, a driving pump 7, and a valve 8. The hollow ball 6 and the driving pump 7 are respectively connected in parallel to the fluid pipe 3 through the valve 8, and then connected to the pressure chamber 4 between the inside of the housing 2 of the one-way pumping device and the deformable pipe 1; The valve 8 is opened or closed to select the hollow ball 6 or the driving pump 7 to drive the blood pump to work.

FIG. 12 is a cross-sectional schematic diagram of the interior of one of the driving pumps of the artificial heart according to Embodiment 5 shown in FIG. 11; The driving pump 7 includes a power supply 70 connected to a control board 71, the control board 71 connected to a motor 72, an output shaft of the motor 72 connected to a rotating wheel 73, the rotating wheel 73 movably connected to a connecting rod 74, the connecting rod 74 movably connected to a reciprocating rod 76, the reciprocating rod 76 connected to a diaphragm 77 through a sliding sleeve 79. The diaphragm 77 has elasticity, and the edge of the diaphragm 77 is fixedly arranged in a spherical pump chamber 78. The pump chamber 78 is connected to the fluid pipe 3. A closed cavity is formed between the diaphragm 77 and the side of the spherical pump chamber 78 facing the fluid pipe 3. An air hole 75 is arranged on the side of the spherical pump chamber 78 where the diaphragm 77 is located and which faces the reciprocating rod 76; The power supply 70 provides power to the control board 71, the control board 71 controls the operation of the motor 72, a rotating wheel 73 is arranged on the output shaft of the motor 72, the motor 72 rotates to drive the rotating wheel 73 to rotate, the rotating wheel 73 drives the reciprocating rod 76 to generate reciprocating motion through a movably connected connecting rod 74 arranged in an offset manner, and the reciprocating rod 76 drives the diaphragm 77 fixed to it to reciprocate. Since the diaphragm 77 and the inside of the pump chamber 78 form a closed space connected to the fluid pipe 3, the reciprocating motion of the diaphragm 77 drives the fluid inside the pump chamber 78 to flow back and forth through the fluid pipe 3. The fluid pipe 3 transmits the fluid to act on the one-way pumping device, forming a pulsating pressure change in the pressure chamber 4, and finally acting on the deformable pipe 1 to drive the blood inside it to flow.

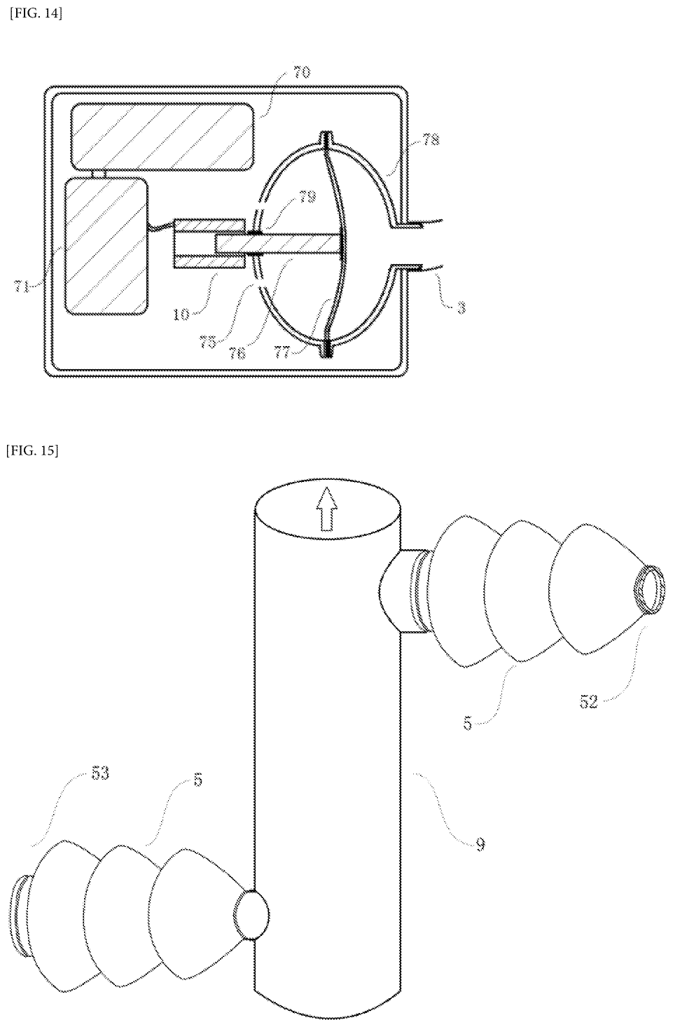

FIG. 14 is a cross-sectional schematic diagram of the interior of the second driving pump of the artificial heart according to Embodiment 5 shown in FIG. 11; The driving pump 7 includes a power supply 70 connected to a control board 71, the control board 71 connected to an electromagnetic coil 10, one end of a reciprocating rod 76 extending into the electromagnetic coil 10, the reciprocating rod 76 connected to a diaphragm 77 through a sliding sleeve 79. The diaphragm 77 has elasticity, and the edge of the diaphragm 77 is fixedly arranged in a spherical pump chamber 78. The pump chamber 78 is connected to the fluid pipe 3. A closed cavity is formed between the diaphragm 77 and the side of the spherical pump chamber 78 facing the fluid pipe 3. An air hole 75 is arranged on the side of the spherical pump chamber 78 where the diaphragm 77 is located and which faces the reciprocating rod 76. At least the part of the reciprocating rod 76 inside the electromagnetic coil 10 has ferromagnetism; The difference between the embodiment shown in this figure and the embodiment shown in FIG. 12 lies in the different driving pump parts, and their working principles are the same, which will not be repeated here. This embodiment uses the electromagnetic coil 10 to drive the reciprocating rod 76. At least the part of the reciprocating rod 76 inside the electromagnetic coil 10 is made of ferromagnetic material, and other parts of the reciprocating rod 76 can be made of materials such as aluminum alloy or plastic.

FIG. 13 is a schematic diagram of the structure of a one-way pumping device according to Embodiment 6 of this application; The difference from FIG. 9 is that the deformable pipe 1 shown in the figure of this embodiment is in a corrugated pipe shape, and other parts are arranged the same as those shown in FIG. 9.

The artificial heart can also be provided with a blood pressure detection device, a pulse detection device, etc., which are connected to the control board to control the operation of each part. These are contents known and set by those skilled in the art, and will not be described in detail here.

FIG. 15 is a schematic diagram of the structure of an exhaust gas supercharging device for an internal combustion engine of this application; An exhaust gas supercharging device for an internal combustion engine, the internal combustion engine body is provided with an exhaust pipe 9. The one-way pumping device is arranged on the exhaust pipe 9. The deformable pipe 1 of the one-way pumping device is arranged inside the exhaust pipe 9. The one-way pipes 5 at both ends of the deformable pipe 1 extend out through the outer wall of the exhaust pipe 9. Both ends of the one-way pumping device are connected in series to the intake pipe of the internal combustion engine. The reverse inlets 52 at both ends of the one-way pumping device face the direction of the internal combustion engine body, and the forward inlets 53 face the direction of the intake port of the intake pipe of the internal combustion engine; The upward arrow in FIG. 15 indicates the flow direction of the exhaust gas.

The one-way pipes 5 at both ends of the one-way pumping device are arranged on the same side of the exhaust pipe 9 or arranged on the side of the exhaust pipe 9 at other angles.

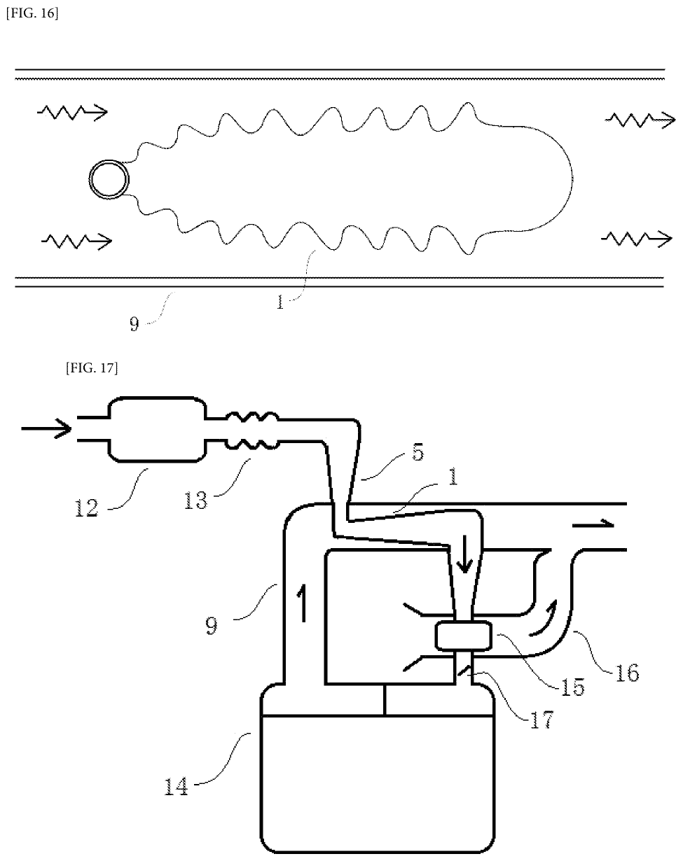

FIG. 16 is a partial cross-sectional schematic diagram of the exhaust pipe shown in FIG. 15; This figure shows that the deformable pipe 1 is arranged inside the exhaust pipe 9. Both ends of the deformable pipe 1 pass through the pipe wall of the exhaust pipe 9 to connect to the one-way pipe 5. The cross-sectional area of the left end of the deformable pipe 1 is smaller than that of the right end. The left end opening of the deformable pipe 1 shown in the figure faces outward, and the right end opening faces inward. The deformable pipe 1 is in a corrugated pipe shape. When the internal combustion engine works, the exhaust gas of the internal combustion engine flows through the periphery of the deformable pipe 1 along the direction indicated by the arrow inside the exhaust pipe 9. Since the exhaust gas of the internal combustion engine is a pulsating fluid, it will generate traveling waves, making the pipe wall of the deformable pipe 1 follow the pulsation, and driving the intake gas of the internal combustion engine inside the deformable pipe 1 to flow in the same direction as the exhaust gas flow. The more energy the exhaust gas has, the more energy is transmitted to the intake gas of the internal combustion engine, thus increasing the intake pressure and intake volume of the internal combustion engine; To solve the problem of high-temperature influence of the exhaust gas of the internal combustion engine, the deformable pipe 1 is made of thin-walled stainless steel, titanium alloy, or other metal materials.

Without affecting the exhaust gas flow rate, the one-way pumping device can be integrally arranged inside the exhaust pipe 9, and both ends are connected to the intake pipe of the internal combustion engine through pipes passing through the wall of the exhaust pipe.

To increase the intake supercharging effect of the internal combustion engine, the length of the deformable pipe 1 can be increased, and one or more brackets 11 are arranged between the deformable pipe 1 and the inner wall of the exhaust pipe 9 to fix the deformable pipe 1 without contacting the inner wall of the exhaust pipe. Both ends of the bracket 11 are fixedly connected to the inner wall of the exhaust pipe 9 and the outer wall of the deformable pipe 1. The bracket 11 has elasticity, for example, the bracket 11 is made of a coil spring or a spring leaf, refer to FIG. 19. For example, the bracket 11 is made of a stainless steel spring.

FIG. 17 is a schematic diagram of the connection structure between one of the exhaust gas supercharging devices for the internal combustion engine and the internal combustion engine of this application; In this figure, the intake port of the intake pipe of the internal combustion engine is provided with a filter 12 and a resonance pipe 13 for shock absorption, which is connected to the one-way pumping device at the back. The one-way pumping device passes through the exhaust pipe 9 and then is connected to the internal combustion engine body 14 through an intercooler 15 and a throttle valve 17. A heat dissipation pipe 16 is arranged on the periphery of the intercooler 15. One end of the heat dissipation pipe 16 is connected to the exhaust pipe 9, and the other end is open to the outside. When the internal combustion engine works, the pulsating exhaust gas in the exhaust pipe 9 flows through the periphery of the deformable pipe 1 of the one-way pumping device, transmits kinetic energy to the deformable pipe 1, and drives the gas inside the deformable pipe 1 to flow in the same direction as the exhaust gas, that is, supercharging the intake gas of the internal combustion engine. The exhaust gas passes through the connection position between the heat dissipation pipe 16 and the exhaust pipe 9. Due to the fast flow speed of the exhaust gas, low pressure is generated to attract the gas inside the heat dissipation pipe 16 to flow, so that the intercooler 15 inside the heat dissipation pipe 16 accelerates heat dissipation. The arrow in the heat dissipation pipe 16 indicates the gas flow direction. The main purpose of this figure is to show the arrangement between the one-way pumping device and the exhaust pipe 9 and the intake pipe of the internal combustion engine. The intake pipe and the exhaust pipe 9 of the internal combustion engine can also be combined with the technical solution provided by this application with various component arrangements, such as a gas flow sensor, a temperature sensor, a pressure relief valve, etc., which belong to the prior art and will not be described in detail here.

The one-way pipes 5 at both ends of the one-way pumping device are arranged on the same side of the exhaust pipe 9 or arranged on the side of the exhaust pipe 9 at other angles.

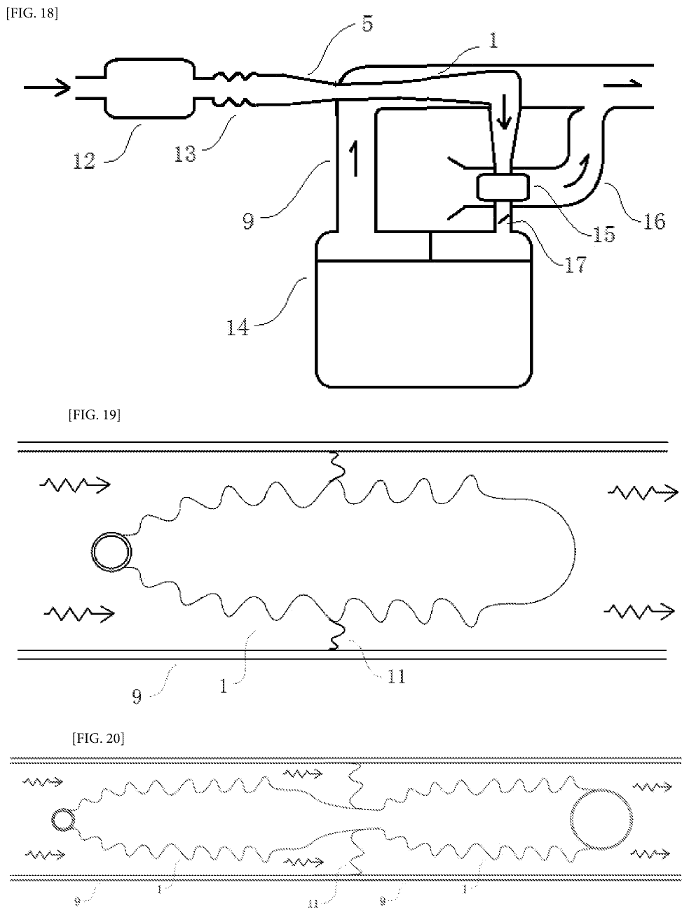

FIG. 18 is a schematic diagram of the connection structure between the second exhaust gas supercharging device for the internal combustion engine and the internal combustion engine of this application; The difference between this figure and FIG. 17 lies in the different arrangement directions of the one-way pipe 5 and the deformable pipe 1 of the one-way pumping device relative to the exhaust pipe 9. In this figure, the one-way pipe 5 at the left end of the one-way pumping device is connected to the deformable pipe 1 in the same axis, while the one-way pipe 5 at the right end is connected at 90 degrees. The purpose of this figure is to show that the one-way pumping device can be arranged at various angles according to actual arrangements.

To increase the intake supercharging effect of the internal combustion engine, a plurality of deformable pipes 1 can be arranged inside the exhaust pipe 9, and the plurality of deformable pipes 1 are connected in series. Refer to FIG. 20. In this figure, two deformable pipes 1 are arranged inside the exhaust pipe 9. The two ends of the deformable pipes shown in the figure are open outward. The right end of the left deformable pipe 1 is connected in series with the left end of the right deformable pipe 1 inside the exhaust pipe 9. The connection between the two deformable pipes 1 is that the end with the larger cross-sectional area of the left deformable pipe 1 is connected to the end with the smaller cross-sectional area of the right deformable pipe 1. After the two deformable pipes 1 are connected as a whole, the cross-sectional area of the left port is smaller than that of the right port. The left end of the left deformable pipe 1 passes through the pipe wall of the exhaust pipe 9 to connect to the one-way pipe 5, and the right end of the right deformable pipe 1 passes through the pipe wall of the exhaust pipe 9 to connect to the one-way pipe 5. An elastic bracket 11 is arranged between the left deformable pipe 1 and the right deformable pipe 1 to limit the position of the deformable pipes 1 inside the exhaust pipe 9. This arrangement increases the length of the deformable pipes 1 inside the exhaust pipe 9, and forms a continuously changing cross-sectional area inside and outside the deformable pipes 1 through the series connection of multiple deformable pipes 1, enhancing the interaction between the pipe wall of the deformable pipes 1 and the exhaust gas inside the exhaust pipe 9, as well as the supercharging effect of the interaction between the pipe wall of the deformable pipes 1 and the intake gas of the internal combustion engine inside the deformable pipes 1. The curved arrow in the figure indicates the flow direction of the pulsating exhaust gas inside the exhaust pipe 9. Under the action of the pulsating exhaust gas, the same direction pulsating driving force is generated for the fluid in the connected deformable pipes 1.

An exhaust gas supercharging device for an internal combustion engine. The internal combustion engine body is provided with an exhaust pipe 9. A plurality of the one-way pumping devices are arranged on the exhaust pipe 9. The deformable pipes 1 of the plurality of one-way pumping devices are arranged inside the exhaust pipe 9. Both ends of the plurality of one-way pumping devices are respectively connected to different pipes to supercharge the fluid inside them. It includes at least two of the following: both ends of one of the one-way pumping devices are connected in series to the intake pipe of the internal combustion engine to supercharge the intake air; both ends of one of the one-way pumping devices are connected to the air pressure pipe of the vehicle to supercharge the air inside the air pressure pipe, replacing the air pump in the prior art; both ends of one of the one-way pumping devices are connected to the fuel pipe of the vehicle to drive fuel to the internal combustion engine for operation, replacing the fuel pump in the prior art; both ends of one of the one-way pumping devices are connected to the oil pipe of the vehicle to drive oil to the lubrication part, replacing the oil pump in the prior art; by arranging a plurality of one-way pumping devices and using exhaust gas, the kinetic energy of the exhaust gas is used to drive and supercharge different systems, which is used for braking, etc. Those skilled in the art can set devices in the prior art according to the needs of different systems, such as a device for cooling the intake air of the internal combustion engine, a device for cooling the fuel of the internal combustion engine, etc. Its working principle is the same as that described above in this application, and will not be repeated here.

The plurality of one-way pumping devices can be set to different sizes and at different positions of the exhaust pipe 9 according to actual needs. For example, the deformable pipes 1 of two one-way pumping devices are arranged in parallel inside the exhaust pipe 9, or the deformable pipes 1 of two one-way pumping devices are arranged in the front and rear along the axial direction of the exhaust pipe 9 inside the exhaust pipe 9.

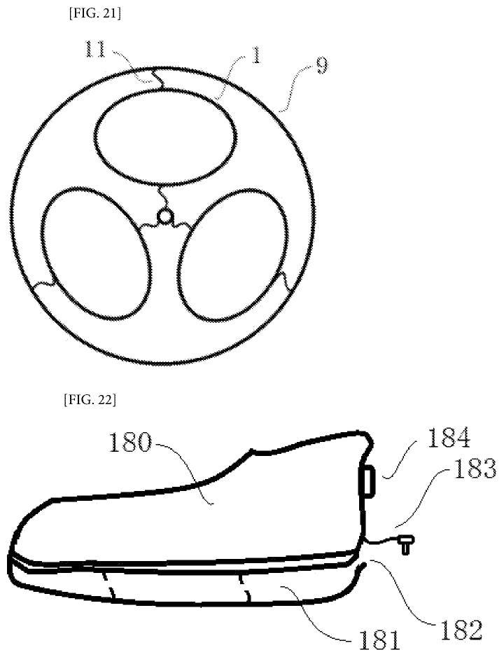

FIG. 21 is a schematic diagram of the deformable pipes arranged in parallel inside the exhaust pipe according to Embodiment 8 of this application; In this figure, three deformable pipes 1 of the one-way pumping device are arranged in parallel inside the exhaust pipe 9. The three deformable pipes 1 are connected to the inner wall of the exhaust pipe 9 through elastic brackets 11 and connected to each other to limit their positions. The three deformable pipes 1 are respectively connected to pipes with the same or different functions outside the exhaust pipe 9. For example, both ends of the one-way pumping devices to which the three deformable pipes 1 belong are connected in series to the intake pipe of the internal combustion engine to supercharge the intake air of the internal combustion engine. The parallel arrangement of multiple deformable pipes 1 can increase the contact area between the exhaust gas inside the exhaust pipe 9 and the deformable pipes 1 compared with a single large-sized deformable pipe 1, thereby increasing the kinetic energy transfer between them; In addition, the cross-section of the deformable pipe 1 in this figure is elliptical. Setting the deformable pipe 1 to have an elliptical cross-section is beneficial to increase the contact area with the outside, facilitate the parallel arrangement of multiple deformable pipes 1, and facilitate the shape change of the deformable pipes 1. The elliptical cross-section is more likely to undergo elastic deformation along the radial direction of the cross-section when subjected to an external force than the circular cross-section; The three deformable pipes 1 are respectively connected to pipes with different functions outside the exhaust pipe 9. For example, both ends of the one-way pumping devices to which the three deformable pipes 1 belong are respectively connected to the intake pipe, fuel supply pipe, and oil pipe of the internal combustion engine to supercharge the fluid inside the three pipes.

The three deformable pipes 1 shown in FIG. 21 are arranged in parallel. The three deformable pipes 1 can be arranged axially inside the exhaust pipe 9 respectively. The three deformable pipes 1 are respectively connected to pipes with the same or different functions outside the exhaust pipe 9. Those skilled in the art can understand and implement according to the description of this application, and the specific structural embodiment figures will not be shown here.

FIG. 22 is a cross-sectional schematic diagram of the airbag shoe according to Embodiment 9 of this application; The airbag shoe 18 is used for the external part of the artificial heart. The airbag shoe 18 includes a shoe body 180 and an airbag 181 arranged under the shoe body 180. The airbag 181 is provided with an airbag port 182 at the heel position of the shoe body 180. Squeezing and releasing the airbag 181 deforms it, and the internal gas enters and exits through the airbag port 182. An airbag seal 183 is arranged on the shoe body 180 near the airbag port 182. The airbag seal 183 can seal the airbag inlet/outlet 182. The airbag seal 183 and the airbag port 182 can be sealed by means of a snap-fit or screw connection. The airbag 181 has elasticity and is filled with air in a free state. The airbag port 182 is sealed with the airbag seal 183. The airbag 181 has a shock absorption effect under the shoe body 180. The airbag seal 183 is removed from the airbag port 182, and the fluid pipe 3 is inserted into the airbag port 182 to connect to the airbag 181. The fluid pipe 3 is fixed with a pipe clip 184. The fluid pipe 3 extends upward along the human leg and is connected to the pressure chamber 4 of the one-way pumping device. When a person walks, the airbag 181 is squeezed and released by the weight of the human body, so that the internal gas transmits pressure changes to the fluid in the pressure chamber 4 through the fluid pipe 3, driving the one-way pumping device to work; Its working principle is the same as that of the above-mentioned embodiment of this application, and will not be repeated here; The materials and structures of the airbag 181 of the airbag shoe 18 other than those described in this application can adopt the prior art, and will not be described in detail here.

It can be understood that the size ratio of each component in the embodiments described in this application is not completely drawn according to the actual ratio, and those skilled in the art can understand and implement the relevant content. The directional terms such as left and right described in this application are used to explain and understand the drawings and the structure of this application, and are not used to limit the technical solution of this application.

The principles and structures known to those skilled in the art other than the above contents described in this application will not be described in detail here.

The shapes of the drawings in the embodiments of the present application are used to illustrate the application of the present application. The present application has only been described through selected embodiments; therefore, it is obvious that the above-described embodiments are for illustration rather than limiting the present application.

1. A one-way pumping device, wherein the one-way pumping device comprises two one-way pipes and a deformable pipe connected between the two one-way pipes; the two one-way pipes are arranged in one direction; the deformable pipe has radial or/and axial elasticity; the one-way pipe comprises at least one one-way pipe unit, and the one-way pipe unit comprises a main pipe wall, a shunt pipe and a bracket; the main pipe wall is in the shape of a hollow cone with a central through hole, being thin at one end and thick at the other end; the shunt pipe is in the shape of a horn with a central through hole, being thin at one end and thick at the other end; the shunt pipe is suspended and fixed inside the main pipe wall through the bracket, and the shunt pipe is arranged in the same direction as the corresponding main pipe wall, and the shunt pipe is arranged coaxially with the main pipe wall; the central through holes of the shunt pipe and the main pipe wall form a main channel; a shunt channel with a U-shaped cross-section is formed between the shunt pipe and the main pipe wall.

2. The one-way pumping device according to claim 1, wherein when an external force is applied, the pipe wall at the end of the deformable pipe close to the outlet is more prone to deformation than the pipe wall at the end close to the inlet.

3. The one-way pumping device according to claim 1, wherein a housing is further arranged on the periphery of the deformable pipe; a pressure chamber with a closed space is formed between the housing and the deformable pipe; the pipe wall of the housing is provided with at least one fluid pipe, and the fluid pipe is connected to the pressure chamber, the housing is a rigid shell or has elasticity; when the housing has elasticity, under the action of the same external force, the housing is more difficult to change its shape than the deformable pipe.

4. The one-way pumping device according to claim 1, wherein the deformable pipe is a corrugated pipe; the cross-sectional area of the inlet of the deformable pipe is smaller than that of the outlet; the cross-section of the deformable pipe is circular or elliptical.

5. An artificial heart, comprising an extracorporeal part and an intracorporeal part; the intracorporeal part is used to partially or fully replace the function of the heart, and the intracorporeal part is a blood pump; the extracorporeal part is connected to the intracorporeal part through a pipeline for driving and controlling the intracorporeal part, wherein the blood pump adopts the one-way pumping device according to claim 3; the blood pump is connected to the extracorporeal part through a fluid pipe; the extracorporeal part generates pulsating pressure, which transmits power to the fluid in the pressure chamber of the one-way pumping device through the fluid pipe, so that the fluid in the pressure chamber generates pulsating pressure to drive the deformable pipe to change its shape, thereby changing the volume of the deformable pipe; the change in the internal volume of the deformable pipe, together with the action of the one-way pipe, enables blood to flow unidirectionally inside the one-way pumping device.

6. The artificial heart according to claim 5, wherein the extracorporeal part is a hollow sphere, and the hollow sphere is connected to the pressure chamber between the housing of the one-way pumping device and the deformable pipe through a fluid pipe.

7. The artificial heart according to claim 5, wherein the extracorporeal part comprises a hollow sphere, a driving pump and a valve; the hollow sphere and the driving pump are respectively connected in parallel to the fluid pipe through the valve, and then connected to the pressure chamber inside the deformable pipe of the one-way pumping device.

8. The artificial heart according to claim 7, wherein the driving pump comprises a power supply connected to a control board; the control board is connected to a motor; the output shaft of the motor is connected to a rotating wheel; the rotating wheel is movably connected to a connecting rod; the connecting rod is movably connected to a reciprocating rod; the reciprocating rod is connected to a diaphragm through a sliding sleeve; the diaphragm has elasticity, and the edge of the diaphragm is fixedly arranged in a spherical pump cavity; the pump cavity is connected to the fluid pipe; a closed cavity is formed between the diaphragm and the side of the spherical pump cavity facing the fluid pipe, an air hole is arranged on the side of the spherical pump cavity where the diaphragm is arranged and which faces the reciprocating rod.

9. The artificial heart according to claim 7, wherein the driving pump comprises a power supply connected to a control board; the control board is connected to an electromagnetic coil; one end of a reciprocating rod extends into the electromagnetic coil; the reciprocating rod is connected to a diaphragm through a sliding sleeve; the diaphragm has elasticity, and the edge of the diaphragm is fixedly arranged in a spherical pump cavity; the pump cavity is connected to the fluid pipe; a closed cavity is formed between the diaphragm and the side of the spherical pump cavity facing the fluid pipe; an air hole is arranged on the side of the spherical pump cavity where the diaphragm is arranged and which faces the reciprocating rod; at least the part of the reciprocating rod inside the electromagnetic coil is ferromagnetic.

10. The artificial heart according to claim 5, wherein the extracorporeal part comprises an airbag shoe; the airbag shoe comprises a shoe body and an airbag arranged under the shoe body; the airbag is provided with an airbag port at the heel position of the shoe body; the airbag is squeezed and released to deform it, and the internal gas enters and exits through the airbag port; an airbag seal is arranged on the shoe body near the airbag port; the airbag has elasticity; the fluid pipe is inserted into the airbag port to connect to the airbag, and the fluid pipe is fixed with a pipeline clip; the fluid pipe extends upward along the human leg and is connected to the pressure chamber of the one-way pumping device.

11. A device for exhaust gas supercharging of an internal combustion engine, the internal combustion engine body is provided with an exhaust pipe, wherein a one-way pumping device is arranged in the exhaust pipe; the one-way pumping device comprises two one-way valves and a deformable part connected between the two one-way valves; the deformable part is a pipe with elasticity; the two one-way valves are arranged in one direction; the exhaust gas of the internal combustion engine with changing pressure passes through the outside of the deformable part and acts on the deformable part to change its internal volume; due to the action of the one-way valves, the fluid inside the one-way pumping device flows unidirectionally.

12. The device for exhaust gas supercharging of an internal combustion engine according to claim 11, wherein one or more one-way pumping devices comprises two one-way pipes and a deformable pipe connected between the two one-way pipes; the two one-way pipes are arranged in one direction; the deformable pipe has radial or/and axial elasticity; the one-way pipe comprises at least one one-way pipe unit, and the one-way pipe unit comprises a main pipe wall, a shunt pipe and a bracket; the main pipe wall is in the shape of a hollow cone with a central through hole, being thin at one end and thick at the other end; the shunt pipe is in the shape of a horn with a central through hole, being thin at one end and thick at the other end; the shunt pipe is suspended and fixed inside the main pipe wall through the bracket. and the shunt pipe is arranged in the same direction as the corresponding main pipe wall, and the shunt pipe is arranged coaxially with the main pipe wall; the central through holes of the shunt pipe and the main pipe wall form a main channel; a shunt channel with a U-shaped cross-section is formed between the shunt pipe and the main pipe wall
are arranged in the exhaust pipe; both ends of the one-way pumping device are connected in series to the intake pipeline of the internal combustion engine; at least the deformable pipe of the one-way pumping device is arranged inside the exhaust pipe; the reverse inlets at both ends of the one-way pumping device face the direction of the internal combustion engine body, and the forward inlets face the direction of the air inlet of the intake pipeline of the internal combustion engine.

13. The device for exhaust gas supercharging of an internal combustion engine according to claim 12, wherein a plurality of the one-way pumping devices are arranged in the exhaust pipe; the deformable pipes of the plurality of one-way pumping devices are arranged inside the exhaust pipe; the plurality of deformable pipes are arranged in parallel along the axial direction (longitudinally) or/and radial direction of the exhaust pipe inside the exhaust pipe; both ends of the plurality of one-way pumping devices are respectively connected in series to the intake pipeline of the internal combustion engine.

14. The device for exhaust gas supercharging of an internal combustion engine according to claim 12, wherein a plurality of the one-way pumping devices are arranged in the exhaust pipe; the deformable pipes of the plurality of one-way pumping devices are arranged inside the exhaust pipe; the plurality of deformable pipes are arranged in parallel along the axial direction (longitudinally) or/and radial direction of the exhaust pipe inside the exhaust pipe; both ends of the plurality of one-way pumping devices are respectively connected in series to different pipelines of the internal combustion engine, which include at least two of the following: both ends of one of the one-way pumping devices are connected to the intake pipeline of the internal combustion engine; both ends of one of the one-way pumping devices are connected to the air pressure pipeline of the vehicle; both ends of one of the one-way pumping devices are connected to the fuel pipeline of the vehicle; both ends of one of the one-way pumping devices are connected to the engine oil pipeline of the vehicle.
